(19) United States
(12) Patent Application Publication
(10) Pub. No.: US 2023/0263999 A1
(43) Pub. Date: Aug. 24, 2023

(54) SYSTEMS AND METHODS OF RESTORING PERFUSION TO A VESSEL

(71) Applicant: Neuravi Limited, Galway (IE)

(72) Inventors: David VALE, Barna (IE); Brendan CASEY, Galway (IE); Michael GILVARRY, Headford (IE); Jacqueline O'GORMAN, Clare (IE); Daniel KING, Galway (IE); Kevin McARDLE, Loughrea (IE); Brian FAHY, Galway (IE); Mairsil CLAFFEY, Galway (IE); Malgorzata Alicja KOKOSZKA, Galway (IE)

(73) Assignee: Neuravi Limited, Galway (IE)

(21) Appl. No.: 18/104,611

(22) Filed: Feb. 1, 2023

Related U.S. Application Data
(60) Provisional application No. 63/313,107, filed on Feb. 23, 2022.
(60) Provisional application No. 63/404,432, filed on Sep. 7, 2022.

(51) Int. Cl. A61M 25/10; A61B 17/12

(52) U.S. Cl. CPC A61M 25/10; A61B 17/12109; A61B 17/221

(57) ABSTRACT

A method and/or use of restoring blood flow in neurovasculature by removing thrombus in a plurality of human patients experiencing ischemic stroke. A first stent retriever device is passed by, through, or about a cerebral occlusion in a blood vessel of one of the plurality of human patients. Then a revascularization device is passed by, through, or about the cerebral occlusion in the blood vessel of the one of the plurality of human patients to restore perfusion to the blood vessel and achieve at least approximately 88% final revascularization rate for the plurality of human patients under the modified treatment in cerebral infarction score of equal to or greater than a grade of 2b (mTICI≥2b).

### CROSS-REFERENCE TO RELATED APPLICATIONS

This application claims the benefit of U.S. Provisional Patent Application No. 63/313,107, filed Feb. 23, 2022, which is incorporated by reference herein in its entirety. This application also claims the benefit of U.S. Provisional Patent Application No. 63/404,432, filed Sep. 7, 2022, which is incorporated by reference herein in its entirety.

### FIELD

This disclosure relates to devices and methods of removing acute blockages from blood vessels.

### BACKGROUND

The World Health Organization estimates that 15,000,000 blood clots occur annually. Clots may develop and block vessels locally without being released in the form of an embolus—this mechanism is common in the formation of coronary blockages. Acute obstructions may include blood clots, misplaced devices, migrated devices, large emboli and the like. Thromboembolism occurs when part or all of a thrombus breaks away from the blood vessel wall. This clot is then carried in the direction of blood flow. The large vessels of the brain include the Internal Carotid Artery (ICA), Middle Cerebral Artery (MCA), Vertebral Artery (VA), and the Basilar Artery (BA). Clots can include a range of morphologies and consistencies. Long strands of softer clot material may tend to lodge at bifurcations or trifurcations, resulting in multiple vessels being simultaneously occluded over significant lengths. Older clot material can also be less compressible than softer fresher clots, and under the action of blood pressure it may distend the compliant vessel in which it is lodged. Clots may also vary greatly in length, even in any one given area of the anatomy. For example, clots occluding the middle cerebral artery of an ischemic stroke patient may range from just a few millimeters to several centimeters in length.

Of the 15,000,000 clots that occur annually, one-third of patients die and another one-third are disabled. Two of the primary factors associated with mortality in these patients are the occlusion location and the time to treatment. Large-vessel occlusions present in 46% of unselected acute stroke patients presenting in academic medical centers, are associated with higher stroke severity. These more proximal vessels feed a large volume of brain tissue, ergo clinicians use the presenting NIHSS (National Institute of Health Stroke Scale) score as an indicator of large-vessel occlusion.

With this, it is understood that an ischemic stroke may result if the clot lodges in the cerebral vasculature. It is estimated that 87% of stroke cases are acute ischemic stroke (AIS). In the United States alone, roughly 700,000 AIS cases occur every year and this number is expected to increase with an ageing population. Occlusion of these large arteries in ischemic stroke is associated with significant disability and mortality. Revascularization of intracranial artery occlusions is the therapeutic goal in stroke therapy. Endovascular mechanical revascularization (thrombectomy) is an increasingly used method for intracranial large vessel recanalization in acute stroke. Currently, a number of mechanical recanalization devices are in clinical use. First generation devices included the Merci Retriever device. Newer devices based on stent-like technology, referred to as “stentrievers” or “stent-retrievers”, are currently displacing these first generation thrombectomy devices for recanalization in acute ischemic stroke.

There are significant challenges associated with designing clot removal devices that can deliver high levels of performance. There are also a number of access challenges that make it difficult to deliver devices. For example, the vasculature in the area in which the clot may be lodged is often fragile and delicate and neurovascular vessels are more fragile than similarly sized vessels in other parts of the body and are in a soft tissue bed. Excessive tensile forces applied on these vessels could result in perforations and hemorrhage. Pulmonary vessels are larger than those of the cerebral vasculature, but are also delicate in nature, particularly those more distal vessels.

Stent-like clot retriever devices are being increasingly used to remove clots from cerebral vessels of acute stroke patients but such devices are not without disadvantages. A stent-like clot retriever relies on its outward radial force to grip the clot. If the radial force is too low, the device will lose its grip on the clot. If the radial force is too high, the device may damage the vessel wall and may require too much force to withdraw. Such devices that have sufficient radial force to deal with all clot types may therefore cause vessel trauma and serious patient injury, and retrievers that have appropriate radial force to remain atraumatic may not be able to effectively handle all clot types. In this respect, retriever devices may differ in size, shape, and physical properties, such as radial force, as discussed above, ease of deployment, friction, radiopacity and interaction with vessel wall. See, Loh Y, Jahan R, McArthur D. Recanalization rates decrease with increasing thrombectomy attempts. American Journal . . . . 2010 May; 31(5):935-9; and Arai D, Ishii A, Chihara H, Ikeda H, Miyamoto S. Histological examination of vascular damage caused by stent retriever thrombectomy devices, J Neurointery Surg. 2016 October; 8(10):992-5. Some designs have also been based on in-vitro stroke models that incorporate realistic clot analogs derived from animal blood that represent the wide range of human clots retrieved from stroke patients. See, Eugène F, Gauvrit J-Y, Ferré J-C, Gentric J-C, Besseghir A, Ronzière T, et al. One-year MR angiographic and clinical follow-up after intracranial mechanical thrombectomy using a stent retriever device, AJNR Am J Neuroradiol. 2015 January; 36(1):126-32 (18), each of which are incorporated by reference herein in their entirety.

Currently, intravenous (IV) lytics are used for patients presenting up to 4.5 hours after symptom onset. Current guidelines recommend administering IV lytics in the 3-4.5 hour window to those patients who meet the ECASS 3 (European Cooperative Acute Stroke Study 3) trial inclusion/exclusion criteria. Since a large percentage of strokes presenting at hospitals are large vessel occlusions, this is an important clinical challenge to address. Additionally, not all patients may be treated with thrombolytic therapy, and so mechanical thrombectomy is a valuable alternative in patients contraindicated to t-PA (tissue plasminogen activator) or where t-PA treatment was not effective.

Though success rates are high when utilizing mechanical thrombectomy, there are still a proportion of patients for which adequate reperfusion cannot be achieved, certainly, in part, due to the clot not being retrieved. In view of these clear performance disadvantages, further reperfusion and patient outcomes advances in AIS treatment are warranted. Further, there is a need to treat challenging situations where the current stent retrievers are unsuccessful during the first few attempts at clot removal. The solution of this disclosure resolves these and other issues of the art.

### SUMMARY

The subject of this disclosure is the use of a clot revascularization device to treat ischemic stroke for restoring perfusion and/or removing a clot and other obstructions from the neurovascular arteries and veins as well as other vascular beds.

In some examples, a method or use is disclosed to restore blood flow in neurovasculature by removing thrombus in a plurality of human patients experiencing ischemic stroke, the method or use including passing a first stent retriever device by, through, or about a cerebral occlusion in a blood vessel of one of the plurality of human patients; then passing a revascularization device by, through, or about the cerebral occlusion in the blood vessel of the one of the plurality of human patients to restore perfusion to the blood vessel and achieve at least approximately 88% final revascularization rate for the plurality of human patients under the modified treatment in cerebral infarction score of equal to or greater than a grade of 2b (mTICI≥2b).

In some examples, the step of passing the revascularization device comprises retracting the revascularization device, after being passed by, through or about the cerebral occlusion, while pinching the cerebral occlusion.

In some examples, the revascularization device is configured to remove the cerebral occlusion or portions thereof that are fibrin-rich.

In some examples, the plurality of human patients includes at least approximately 50 patients.

In some examples, the plurality of human patients includes at least approximately 80 patients.

In some examples, the method or use includes confirming angiographically, after the step of passing the first stent retriever device, that the revascularization rate is less than a grade of 2b (mTICI<2b).

In some examples, inclusion criteria for the plurality of human patients comprising:
Aged ≥18; and
mRS 0-1 prior to a stroke corresponding to the cerebral occlusion.

In some examples, exclusion criteria for the plurality of human patients comprising:
Patients in observational, natural history, and/or epidemiological studies not involving intervention were eligible;
Confirmation of positive pregnancy test according to site specific standard of care;
Patients who had direct aspiration via syringe/mechanical pump during the first and/or second pass attempt prior to the revascularization device;
All patients with severe hypertension on presentation (SBP>220 mmHg and/or DBP>120 mm Hg). All patients, in whom intravenous therapy with blood pressure medications was indicated, with hypertension that remained severe and sustained despite intravenous antihypertensive therapy (SBP>185 mmHg and/or DBP>110 mmHg);
Known cerebral vasculitis;
Known cancer with life expectancy less than 12 months;
Stenosis, or any occlusion, in a proximal vessel that requires treatment or prevents access to the site of occlusion;
Intracranial stenosis that prevented access to the site of occlusion;
Computed tomography (CT) or Magnetic Resonance Imaging (MRI) evidence of recent/fresh hemorrhage on presentation;
Baseline CT or MRI showing mass effect or intracranial tumor (except small meningioma);
Evidence of dissection in the extra or intracranial cerebral arteries; and
Occlusions in multiple vascular territories (i.e. bilateral anterior circulation, or anterior/posterior circulation).

In some examples, the method or use is performed within approximately 6 hours of stroke symptom onset.

In some examples, the method or use is performed within approximately 8 hours of stroke symptom onset.

In some examples, the method or use is performed within approximately 24 hours of stroke symptom onset.

In some examples, the cerebral occlusion is positioned in an internal carotid artery, a M1 segment and/or a M2 segment of a middle cerebral artery, a vertebral artery, or a basilar artery of the patient, the patient being a human.

In some examples, the revascularization device has a collapsed delivery configuration and an expanded deployed configuration, the revascularization device having a proximal pinch section with a spiral shape comprising a spiral pitch; and a distal section with a barrel shape.

In some examples, the revascularization device has, in the expanded configuration, peaks of the proximal pinch section laterally spaced-apart and when under tension, the method or use including pinching the cerebral occlusion between the peaks. The proximal pinch section can include a plurality of cells defined by struts and crowns connected to corresponding struts and/or crowns, and wherein at least some of the struts and/or crowns of the clot engaging section are aligned with the wave-like form to enhance embedding of clot. In some examples, the proximal pinch section can include one or more clot gripping features. In some examples, the proximal pinch section is substantially curvilinear in the collapsed and expanded configurations. In some examples, the proximal pinch section has a transverse cross section between peaks with both flat and curved sections. In some examples, the proximal pinch section has a flat shape in transverse cross section.

In some examples, a method or use is disclosed to restore blood flow in neurovasculature by removing thrombus in a plurality of human patients experiencing ischemic stroke, the method or use including passing a first stent retriever device by, through, or about a cerebral occlusion in a blood vessel of one of the plurality of human patients; then passing a revascularization device by, through, or about the cerebral occlusion in the blood vessel of the one of the plurality of human patients to restore perfusion to the blood vessel and achieve at least approximately 33.3% FPE (mTICI greater than or equal to 2c) after a first pass.

In some examples, a revascularization device to treat ischemic stroke is disclosed. The device can include a collapsed delivery configuration and an expanded deployed configuration. The device can include a proximal pinch section including a spiral shape including a spiral pitch; and a distal section including a barrel shape. The device, after a first stent retriever device is passed by, through, or about a cerebral occlusion in a blood vessel of a human patient, is configured to achieve at least approximately 88% final revascularization rate under the modified treatment in cerebral infarction score of equal to or greater than a grade of 2b (mTICI≥2b).

In some examples, the revascularization device is configured to remove the cerebral occlusion or portions thereof that are fibrin-rich.

In some examples, the cerebral location of the human patient is located in one of the following locations: a carotid artery, a M1 middle cerebral artery, a M2 middle cerebral artery, a basilar artery, and a vertebral artery.

In some examples, in the expanded configuration, the revascularization device includes peaks of the proximal pinch section are laterally spaced-apart and when under tension the proximal pinch section is configured to pinch the cerebral occlusion between the peaks.

In some examples, the revascularization device includes a shaft extending between a proximal end and a distal end; the proximal pinch section coupled to the distal end of the shaft.

In some examples, the proximal pinch section includes a plurality of cells defined by struts and crowns connected to corresponding struts and/or crowns, and wherein at least some of the struts and/or crowns of the clot engaging section are aligned with the wave-like form to enhance embedding of clot.

In some examples, the proximal pinch section includes one or more clot gripping features.

In some examples, the proximal pinch section is substantially curvilinear in the collapsed and expanded configurations.

In some examples, the proximal pinch section includes a transverse cross section between peaks including both flat and curved sections.

In some examples, the proximal pinch section includes a flat shape in transverse cross section.

In some examples, the device is configured for use within approximately 6 hours of stroke symptom onset.

In some examples, the device is configured for use within approximately 8 hours of stroke symptom onset.

In some examples, the device is configured for use within approximately 24 hours of stroke symptom onset.

In some examples, a revascularization device to treat ischemic stroke is disclosed. The device can include a collapsed delivery configuration and an expanded deployed configuration. The device can include a proximal pinch section including a spiral shape including a spiral pitch; and a distal section including a barrel shape. The device, after a first stent retriever device is passed by, through, or about a cerebral occlusion in a blood vessel of a human patient, is configured to achieve at least approximately 33% FPE (mTICI greater than or equal to 2c) after a first pass.

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 shows a patient catheterized via femoral access with an example clot revascularization device positioned in a cerebral vessel using the arterial system for its delivery.

FIG. 2 shows certain anatomy of cerebral arteries above the aortic arch leading to the brain.

FIG. 3 shows an isometric view of an example device of this disclosure.

FIG. 4 shows a representative overview of an example of a study flow used for this disclosure.

FIG. 5 is a table summarizing how subjects were managed in the study of this disclosure.

FIG. 6 is a table summarizing Thrombolysis in Cerebrovascular Infarction (mTICI) inclusive of the 2c rating for the study of this disclosure.

FIG. 7 is a table summarizing Heidelberg Bleeding Classification for the study of this disclosure.

FIG. 8 is a table summarizing classification intensity or severity of adverse events assessed in the study of this disclosure.

FIG. 9 is a table summarizing classification outcomes of adverse events assessed in the study of this disclosure.

FIGS. 10A and 10B illustrate procedural characteristics of the study of this disclosure.

FIGS. 11A, 11B, and 12 illustrate baseline characteristics of the study of this disclosure.

FIG. 13 illustrates revascularization results of the study of this disclosure.

FIG. 14 depicts a graphical overview of one method or use of treating ischemic stroke according to this disclosure.

FIG. 15 depicts a graphical overview of one method or use of treating ischemic stroke according to this disclosure.

FIG. 16 is a table summarizing clot composition assessed in the study of this disclosure.

FIG. 17 illustrates clot composition across all passes, total composition displayed for each subject and sorted by red blood cell content as a result of the study of this disclosure.

FIG. 18 illustrates clot composition across all passes, total composition displayed for each subject and sorted by fibrin content as a result of the study of this disclosure.

FIG. 19 illustrates composition of clot retrieved in first procedural pass of device 200 compared to clot retrieved with comparative device of the study of this disclosure.

### DETAILED DESCRIPTION

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. By “comprising” or “containing” or “including” it is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology were resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

As discussed herein, vasculature of a “subject” or “patient” may be vasculature of a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to a human (e.g., rat, dog, pig, monkey, or the like). It should be appreciated that the subject may be any applicable human patient, for example.

As discussed herein, “operator” may include a doctor, surgeon, or any other individual or delivery instrumentation associated with delivery of a clot revascularization device to the vasculature of a subject.

As discussed herein, “thrombus” can be understood as a clot in the circulatory system that remains in a site of the vasculature hindering or otherwise obstructing flow in a blood vessel. The terms, “clot”, “thrombus”, “obstruction”, “occlusion”, “blockage”, and/or the like, can be and are often used interchangeably throughout this disclosure.

Delivery of a “revascularization device” is typically accomplished via delivery of one or more catheters into the femoral artery and/or the radial artery, guided into the arteries of the brain, vascular bypass, angioplasty, and/or the like. “Revascularization devices” can include, but not be limited to, one or more stents, stentrievers, clot removal devices, clot revascularization devices, aspiration systems, one or more combinations thereof, and/or the like, each of which are often used interchangeably throughout this disclosure.

As discussed herein, “mTICI” means modified thrombolysis in cerebral infarction (TICI) score. An mTICI score of 0 means no perfusion. An mTICI score of 1 means antegrade reperfusion past the initial occlusion but limited distal branch filling with little or slow distal reperfusion. An mTICI score of 2 generally means incomplete antegrade reperfusion wherein the contrast passes the occlusion and opacifies the distal arterial bed but there are residual antegrade perfusion deficits. More particularly, an mTICI score of 2a means antegrade reperfusion of less than half of the occluded target artery previously ischemic territory (e.g., in 1 major division of the MCA and its territory). An mTICI score of 2b means antegrade reperfusion of more than half of the previously occluded target artery ischemic territory (e.g., in 2 major divisions of the MCA and their territories). An mTICI score of 2c means antegrade reperfusion of >90% but less than TICI 3 or near complete reperfusion. An mTICI score of 3 means full perfusion with filling of all distal branches.

It is noted, however, that other measures of cerebral scoring standards, such as expanded TICI (eTICI), other known and/or to-be-developed cerebral scoring standards, provide measures of cerebral scoring and are thus directly and/or indirectly applicable in understanding scope of the presently disclosed solution. eTICI scale is a 7-point compilation of TICI grades that reflects all previously reported thresholds used to define reperfusion after endovascular stroke therapy. For example, eTICI grade 0, just as mTICI, can be equivalent to no reperfusion or 0% filling of the downstream territory. eTICI 1 can indicate thrombus reduction without any reperfusion of distal arteries, including reperfusion of less than half or 1-49%. eTICI of 2b50 can be 50-66% reperfusion. eTICI 2b67 can be 67-89% reperfusion, exceeding TICI but below TICI2C. eTICI 2c can be equivalent to TICI 2C or 90-99% reperfusion. eTICI 3 can be complete or 100% reperfusion, such as TICI 3. It is understood that one of ordinary skill in the art can also correlate between currently known cerebral scoring standards and/or to-be-developed cerebral scoring standards (e.g., from mTICI to eTICI).

As discussed herein, “NIHSS Score” means The National Institutes of Health Stroke Scale, or NIH Stroke Scale (NIHSS) and is a tool used by healthcare providers to objectively quantify the impairment caused by a stroke. The NIHSS is composed of 11 items, each of which scores a specific ability between a 0 and 4. For each item, a score of 0 typically indicates normal function in that specific ability, while a higher score is indicative of some level of impairment. The individual scores from each item are summed in order to calculate a patient's total NIHSS score. The maximum possible score is 42, with the minimum score being a 0.

As discussed herein, “mRS” means the modified Rankin Scale (mRS) that is a commonly used scale for measuring the degree of disability or dependence in the daily activities of people who have suffered a stroke or other causes of neurological disability. The mRS scale runs from 0-6, running from perfect health without symptoms to death. An mRS score of 0 is understood as no symptoms being observed. An mRS score of 1 is understood as no significant disability is observed and the patient is able to carry out all usual activities, despite some symptoms. An mRS score of 2 is understood as slight disability and the patient is able to look after own affairs without assistance, but unable to carry out all previous activities. An mRS score of 3 is understood as moderate disability whereby the patient can require some help but is able to walk unassisted. An mRS score of 4 is understood as moderate severe disability and the patient is unable to attend to own bodily needs without assistance or walk unassisted. An mRS score of 5 is understood as severe disability and the patient requires constant nursing care and attention, bedridden, incontinent. An mRS score of 6 is understood as the patient being deceased.

As discussed herein, the term “safety”, as it relates to a clot revascularization device, delivery system, or method of treatment refers to a relatively low severity of adverse events, including adverse bleeding events, infusion or hypersensitivity reactions. Adverse bleeding events can be the primary safety endpoint and include, for example, major bleeding, minor bleeding, and the individual components of the composite endpoint of any bleeding event.

As discussed herein, unless otherwise noted, the term “clinically effective” (used independently or to modify the term “effective”) can mean that it has been proven by a clinical trial wherein the clinical trial has met the approval standards of U.S. Food and Drug Administration, EMEA or a corresponding national regulatory agency. For example, a clinical study may be an adequately sized, randomized, double-blinded controlled study used to clinically prove the effects of the reperfusion device and related systems of this disclosure. Most preferably to clinically prove the effects of the reperfusion device with respect to an ischemic event, for example, to achieve a clinically effective outcome in for the patient suffering the ischemic event (e.g., mRS less than or equal to 2) and/or achieve reperfusion the vessel(s) afflicted by the ischemic event.

As discussed herein, “sICH” is any extravascular blood in the brain or within the cranium associated with clinical deterioration, as defined by an increase of 4 points or more in the score on the NIHSS, or that leads to death and is identified as the predominant cause of the neurologic deterioration. For the purpose of this disclosure, subjects with sICH identified through all post—treatment scans up to the 24-hour time-point (including those performed due to clinical deterioration), were considered in the study discussed herein.

As discussed herein, the term “computed tomography” or CT means one or more scans that make use of computer-processed combinations of many X-ray measurements taken from different angles to produce cross-sectional (tomographic) images (virtual “slices”) of specific areas of a scanned object, allowing the user to see inside the object without cutting. Such CT scans of this disclosure can refer to X-ray CT as well as many other types of CT, such as positron emission tomography (PET) and single-photon emission computed tomography (SPECT). The present disclosure is related to systems, methods and devices restoring perfusion in blood vessels, and in particular occlusions from cerebral vessels.

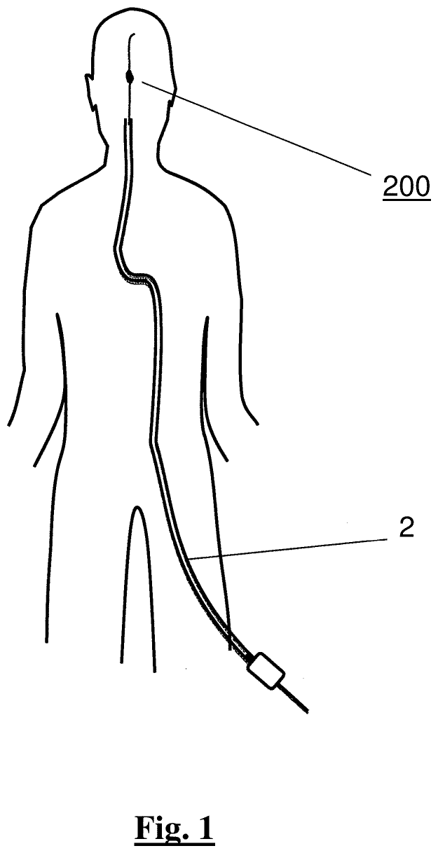

As an example, FIG. 1 depicts a schematic representation of the catheterization of a patient with a clot revascularization device 200, also known as a reperfusion device, via the femoral artery with a catheter 2. Example device 200 is a revascularization device that can restore blood flow in the neurovasculature by removing thrombus in patients experiencing ischemic stroke due to a large vessel neurovascular occlusion (e.g., within 8 hours of symptom onset). Device 200 can be configured for use in the anterior and posterior neurovasculature in vessels of diameter 1.5 mm to 5.0 mm, such as the internal carotid artery, the M1 and M2 segments of the middle cerebral artery, the A1 and A2 segments of the anterior cerebral artery, the basilar, the posterior cerebral and the vertebral arteries. However, it is understood that example device 200 could be used to restore blood flow in less than 8 hours of symptom onset (e.g., 6 hours) or up to 24 hours from symptom onset.

As applicable procedure guidelines change with respect to the use of clot revascularization devices for treatment of ischemic events, it is also conceivable that device 200 could be used more than 24 hours from symptom onset. Device 200 can be understood as including features more clearly described in Appendix 1, as incorporated by reference in its entirety from a U.S. Provisional Application from which this application claims priority, namely U.S. Provisional Application 63/313,107, filed Feb. 23, 2022. Device 200 can also be understood as including features more clearly described in U.S. Pat. Nos. 10,292,723; 10,363,054; 10,617,435; 11,253,278; and 11,147,572, each of which are incorporated by reference in their entirety as if set forth verbatim herein. Note that revascularization devices can also be introduced through the wrist artery (radial access) or directly through the carotid artery. While both radial and carotid access avoids the aortic arches, there are other drawbacks. However, all three approaches are considered to be known to ones of skill in this art.

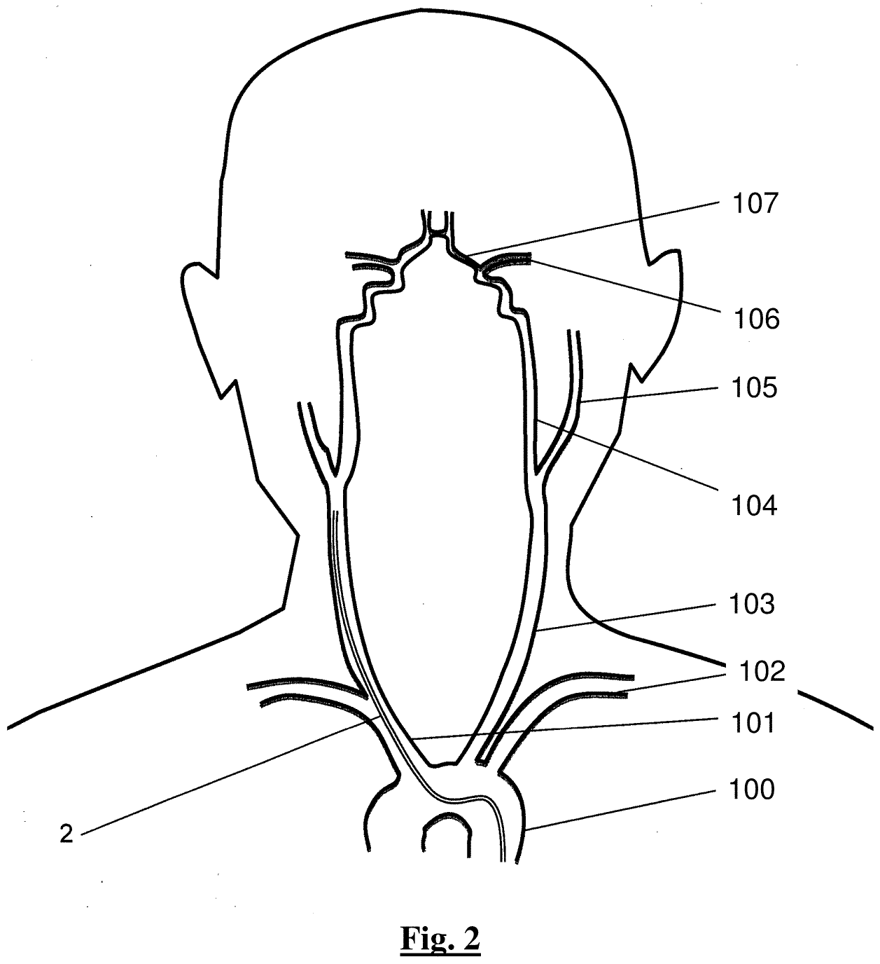

FIG. 2 shows a schematic representation of certain example cerebral vessels. Vessel 100 is the Aorta. Vessel 101 is the brachiocephalic artery. Vessel 102 is the subclavian artery. Vessel 103 is the common carotid artery. Vessel 104 is the internal carotid artery. Vessel 105 is the external carotid artery. Vessel 106 is the middle cerebral artery. Vessel 107 is the anterio-cerebral artery. The catheter 2 from FIG. 1 is shown with its distal end in the common carotid artery. In the more detailed drawings of the invention the details of the access site will not be shown but in general access and delivery is in accordance with FIGS. 1 and 2. Device 200 can be designed for use in the anterior and posterior neurovasculature in vessels such as the internal carotid artery, the M1 and M2 segments of the middle cerebral artery, the vertebral artery, and the basilar arteries. Device 200 can be delivered endovascularly under fluoroscopic guidance in a similar manner to that of other neurovascular clot-retrieval systems.

Once across the site of vessel occlusion, device 200 is deployed to entrap the clot and allow it to be retrieved, hence restoring blood flow. It is understood that device 200 of this disclosure would be used with a delivery system to the site of the clot, including a guide catheter, a microcatheter, and/or a guidewire. It is also contemplated that device 200 of this disclosure could be used in connection with an aspiration system to further facilitate restoring perfusion to the vasculature.

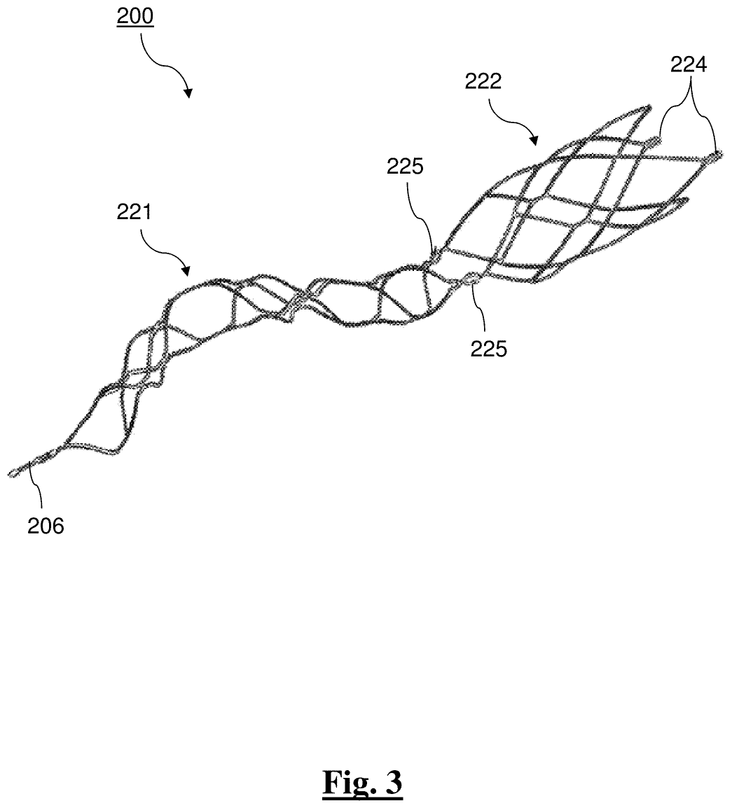

FIG. 3 shows one embodiment of an example clot revascularization device 200 of this disclosure. Device 200 includes a proximal pinch section 221, a distal section 222, distal marker coils 224 and radiopaque markers 225. The proximal pinch section 221 is heat set into a spiral shape, though other shapes of device 200 are contemplated as needed or required. The spiral shape can have a spiral pitch of approximately 14 mm (e.g., within a range of 10-25 mm). The spiral shape can have a spiral outer diameter approximately 5 mm (e.g., within a range of 4.0 to 10 mm). The spiral can form a 360° curve and/or range from 180 to 720°.

A longitudinal center axis of the distal section 222 can be in a barrel shape or otherwise tubular with a lumen and can be offset from a center line of the spiral shape of section 221 to assist in achieving uniform (e.g., low strain) connection between the sections. The distal end of the spiral section is orientated so that it is perpendicular to the proximal face of the barrel section. In this orientation both the struts connecting the spiral section to the barrel section are equal length and have equivalent levels of strain regardless of the cut pattern orientation on the heat forming mandrel. In other iterations the spiral of section 221 can be oriented at an angle to the barrel of section 222.

In some examples, device 200 is configured for removing fibrin rich and/or platelet rich clots. Device 200 can have an expandable structure with a constrained delivery configuration, an expanded clot engaging deployed configuration, and an at least partially constrained clot pinching configuration, whereby at least a portion of the expandable structure is configured to engage the clot in the expanded deployed configuration and to pinch clot on movement from the deployed configuration to the clot pinching configuration. In the clot pinching configuration, device 200 can pinch at least a portion of the clot body as its expandable element is at least partially collapsed from a fully expanded configuration. The expandable element of device 200 can be configured to come into contact with at least a portion of the clot, while maintaining the position of a shaft (e.g., shaft 206) steadfast and effecting pinching substructure of the device so as to pinch at least a portion of the clot and retracting device 200 and the pinched occlusive clot from the patient.

Device 200 can have an elongate shaft 206. Shaft 206 can have a distal end that extends interior of the artery and a proximal end that extends exterior of the artery. Shaft 206 may be a tapered wire shaft, and may be made of stainless steel, MP35N, Nitinol or other material of a suitably high modulus and tensile strength. Shaft 206 can have a coil adjacent its distal end and proximal of the outer member and inner tubular member. The coil may be coated with a low friction material or have a polymeric jacket positioned on the outer surface.

### Study Overview

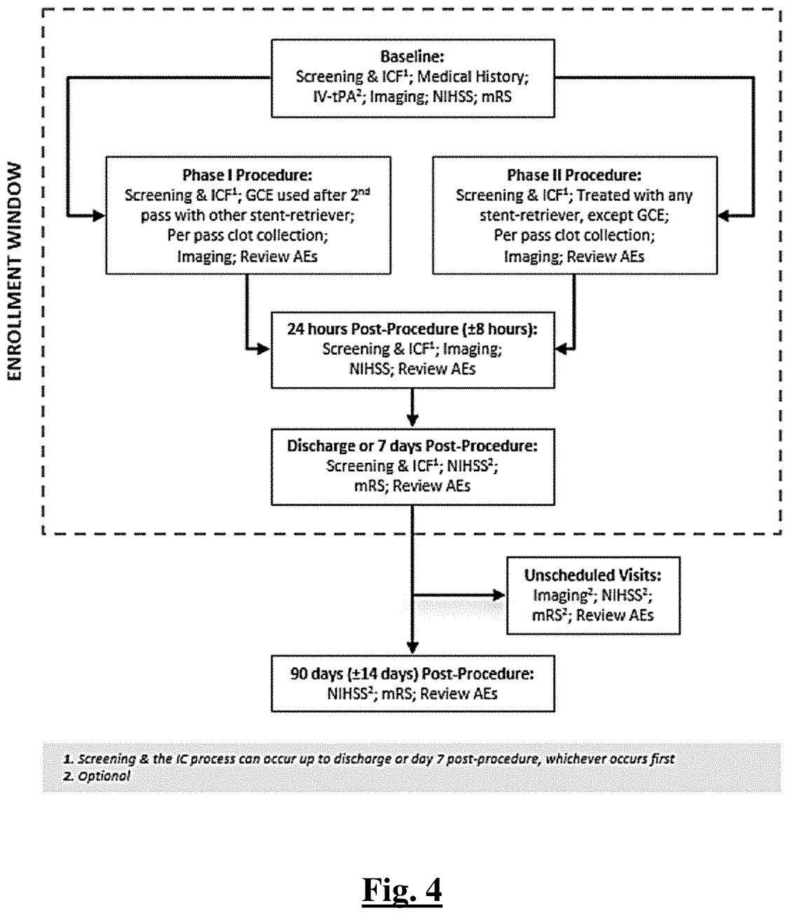

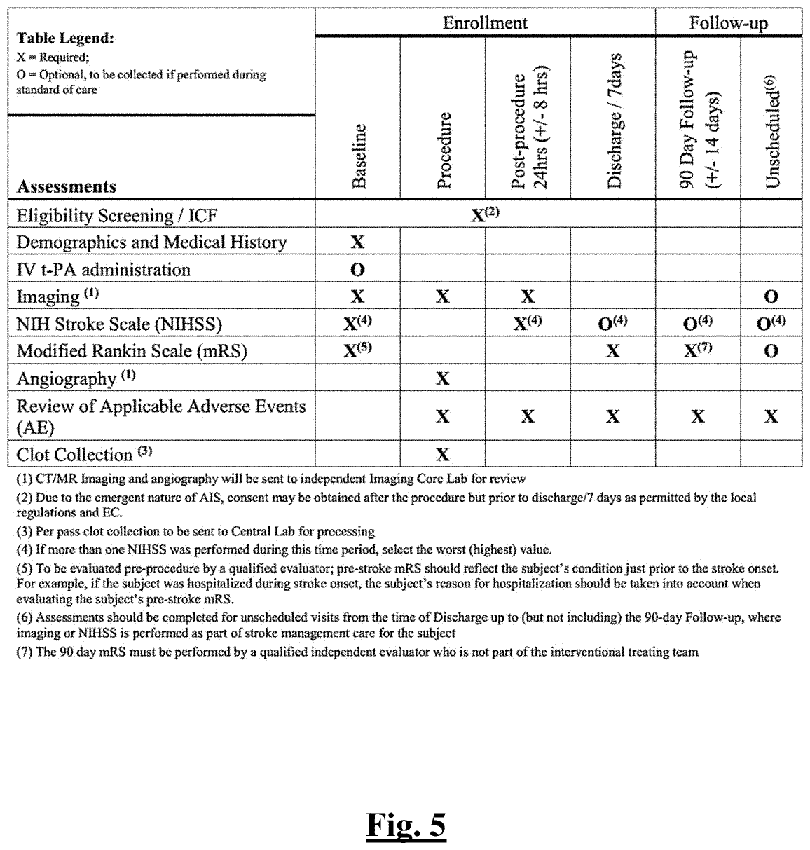

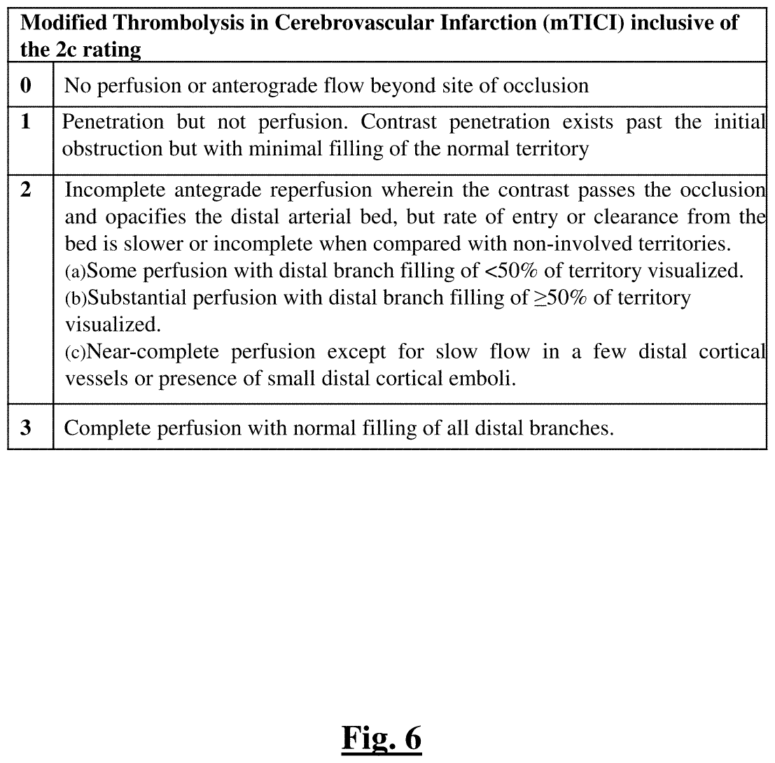

This disclosure is more clearly understood with a corresponding study discussed more particularly below with respect to treatment of ischemic stroke, which is in Appendix 2, as incorporated by reference in its entirety from a U.S. Provisional Application from which this application claims priority, namely U.S. Provisional Application 63/313,107, filed Feb. 23, 2022, which incorporated by reference in its entirety as if set forth verbatim herein. FIG. 4 of this disclosure shows a representative overview of the study flow using the patients of the study with device 200. FIG. 5 is a table summarizing how subjects were managed in the study. Study subjects were managed by the respective Investigators and his/her staff according to the current standard procedures at each participating study site. Data was collected according to the defined schedule listed in FIG. 5. It is understood that data and study information is presented herein for purposes of illustration and should not be construed as limiting the scope of the disclosed technology in any way or excluding any alternative or additional embodiments. The study was a prospective, multi-center, single-arm study that enrolled up to 80 subjects at up to 10 sites. The study evaluated evaluate device 200, post two unsuccessful passes of another stent-retriever, in the treatment of acute ischemic stroke. Data was collected at baseline (prior to thrombectomy), during the procedure, and post-procedure.

Follow up with subjects occurred over 3 months; at 24 hours, 7 days (or discharge), and 90 days post procedure. This study had consecutive serial enrollment of two phases, which led to enrollment of two distinct treatment cohorts in two distinct sequential phases. The objective of this study was to assess the efficacy of device 200 in a real-world setting with patients where the first two passes with another stent-retriever did not achieve an mTICI score of 2b or better. The rate of effectiveness achieved was considered as well as clot characteristics (e.g., Composition of clot components, per pass, Red Blood Cells (RBC), White Blood Cells (WBC), platelets, fibrin and other proteins), as evaluated by the independent Central Lab and clinical outcomes.

The primary endpoint was successful revascularization at the end of the procedure, without rescue as determined by an Independent Core Lab, where the successful revascularization is defined as achieving an mTICI score of 2b or greater. Revascularization was measured using modified Thrombolysis in Cerebrovascular Infarction (mTICI) inclusive of the 2c rating that is described in FIG. 1. Two-sided exact 95% confidence intervals was conducted around the percentage. Subjects who received any rescue therapy prior to the date of 90-day follow-up, were included in the analysis, but considered as not to have achieved this endpoint. Subjects with missing data on mTICI were excluded from the analysis.

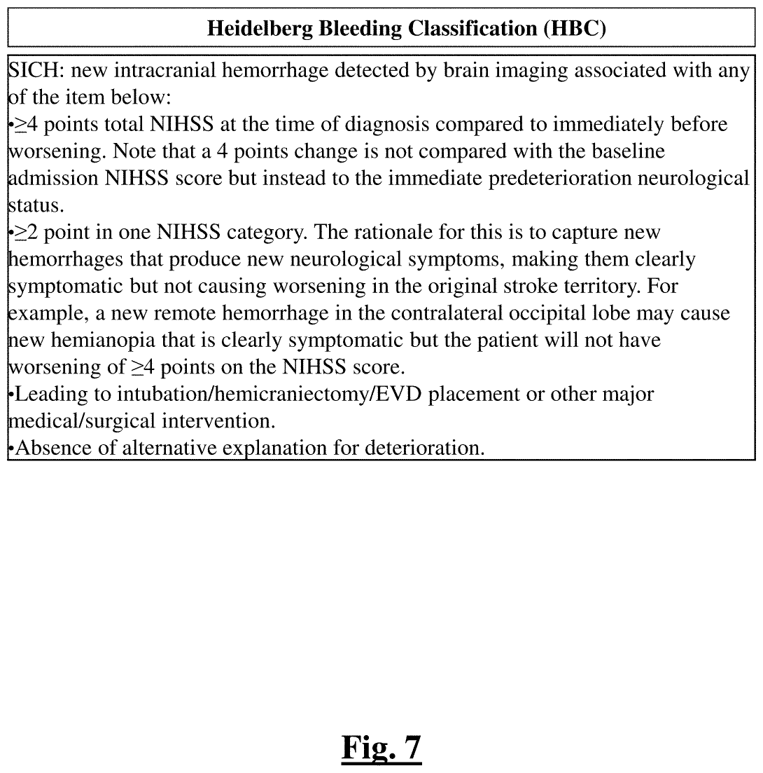

Other endpoints of the study included evaluating successful revascularization (final mTICI≥2c), which was understood as the rate of achieving an mTICI score of 2c or greater at the end of the procedure, as determined by an Independent Core Lab; first study pass (third procedural pass) recanalization (mTICI≥2b), which was understood as the rate of achieving an mTICI score of 2b or greater after the third procedural pass, as determined by an Independent Core Lab with two-sided exact 95% confidence intervals conducted around the percentage; Occurrence of Embolization to a New Territory (ENT), which was understood as the rate of embolization in a previously unaffected territory, following the fifth procedural pass, or the final procedural pass, if earlier; and Symptomatic Intracerebral Hemorrhage (sICH) at 24 hours specified according to the Heidelberg Bleeding Classification (HBC), which was understood as the rate of sICH at 24 hours post-procedure. Symptomatic Intracerebral Hemorrhage (sICH) is defined per the Heidelberg Bleeding Classification, as in FIG. 7.

Other endpoints of the study include 90 Day All-Cause Mortality (e.g., incidence of all mortality regardless of cause at 90 days post-procedure) and mRS of ≤2 at 90 days. Medical Resource Utilization and Health Economics were also evaluated with related data including hospitalization length of stay for index procedure and unscheduled re-hospitalizations as well as healthcare resource utilization for index procedure, post-procedure and re-hospitalizations for unscheduled events. Such information included mean, median, minimum and maximum duration of hospitalization length of stay will be summarized, healthcare resource utilization for index procedure, post-procedure and re-hospitalizations for unscheduled events, and the number and proportion of subjects with re-hospitalizations, and the number of hospitalizations after the index procedure will be summarized.

For the first study pass, this was defined separately by phase. For Phase I, the incidence of recanalization following the first pass of device 200 was analyzed. For Phase II, the incidence of recanalization of the third pass (which was actually the first pass following the initial two passes) of any stent retrievers was analyzed.

For ENT, this likewise depended on phase. For Phase I, the incidence of ENT following the final pass of device 200 was analyzed. For Phase II, the incidence of ENT following the fifth procedural pass, or the final procedural pass, if earlier, of any commercial stent retrievers was analyzed. mRS at 90 days post-procedure was understood as the number and percentage of subjects who reached mRS score of ≤2 at 90 days post-procedure and was analyzed in the mRS Analysis Set population. The two-sided exact 95% confidence interval was conducted around the percentage.

Using standard interventional techniques, access the arterial system and using angiography, the location of the occluded vessel was determined. Then, an appropriate guide catheter, sheath or balloon guide catheter was advanced as close to the occlusion site as possible. Connect a rotating hemostasis valve (RHV) in some examples was connected to the proximal end of this catheter and connect to a continuous flush system. With the aid of a suitable guidewire, and using standard catheterization techniques and fluoroscopic guidance, an appropriately sized microcatheter was advanced up to and across the occlusion so that the distal end of the microcatheter was positioned distal to the occlusion. The guidewire was removed, and device 200 was inserted and advanced into the microcatheter. In some examples, immediately prior to introducing a guide catheter, the physician in the study performed an angiogram of the affected intracranial artery. The purpose of the pre-procedure angiogram was to confirm the location of the occlusion; that the subject remained suitable for treatment with mechanical thrombectomy; and that the subject remains a candidate for the study per the eligibility criteria.

In some examples, device 200 continue to be advanced until radiopaque distal markers of device 200 approach the distal region of the microcatheter. Device 200 was positioned in the clot ideally such that the end of the proximal radiopaque coil was aligned with the proximal face of the clot. To fully deploy device 200 within the clot, the microcatheter was retracted until the distal tip of the microcatheter was positioned over the proximal radiopaque coil of device 200.

The study collected imaging data as assessed by Imaging Core Lab, including Baseline-CT/MR imaging (e.g., Infarct volume, Clot location, Clot length, Clot radiodensity on CT/Susceptibility Vessel Sign (SVS) on MRI), procedural angiography (e.g., mTICI score for every pass, clot location for every pass (proximal face of clot), emboli to new territories), post procedure as to CT/MR imaging (e.g., intracranial hemorrhage). Hemorrhages were classified according to the following categories HI 1—Scattered small petechiae, no mass effect; HI 2—Confluent petechiae, no mass effect; PH1—Hematoma within infarcted tissue, occupying <30%, no substantive mass effect; PH2—Hematoma occupying 30% or more of the infarcted tissue, with obvious mass effect; RIH—Parenchymal hematoma remote from infarcted brain tissue; IVH—Intraventricular Hemorrhage; SAH—Subarachnoid Hemorrhage; and SDH—Sub Dural Hemorrhage.

Prior to clot retrieval, the microcatheter was re-advanced to the clot while holding device 200 push wire static until a predetermined resistance was met. If the operator felt significant resistance, she did not continue to advance. Device 200 was withdrawn with microcatheter slowly and carefully as a single unit to the guide catheter while aspirating through the guide, and maintaining microcatheter and device 200 position relative to each other during the withdrawal step. In some examples, vigorous aspiration was applied by syringe and device 200 withdrawn with microcatheter into the guide catheter and continue to aspirate until device 200 reached the RHV on the guide. The operator could then disconnect the RHV from the guide and remove device 200, microcatheter and RHV together from the guide. Device 200 was used for up to three retrieval attempts. If an additional pass was to be made with device 200, then any captured thrombus was removed from device 200, and device 200 was cleaned in heparinized saline, rubbing gently from proximal to distal to remove any residual thrombus material.

In the event that the subject was excluded as a result of the angiogram, the subject was considered a screen-failure and did not require any study follow-up. Up to the first 50 patients in the study (Phase I) were treated using any commercially available stent-retriever for the first two occlusion retrieval attempts (also known as a pass), and were treated using device 200 from the third pass onward, where eligibility criteria were met. Phase II subjects were not treated with device 200, and instead were treated with any commercially available stent-retriever for the full duration of the procedure. Endovascular treatment with device 200, and the other stent-retrievers, was performed per hospital standard technique and in accordance with the applicable devices' IFU. The following data was captured during the procedure, in Phase I and Phase II, and recorded on the eCRF:
Name of the treating physician;
Type of sedation used;
Vessel location of the occlusion being treated per pass;
mTICI scores per pass (Pre-device deployment and post-device retrieval);
Names and sizes of stent-retrievers used per pass;
Lot number of device 200 used, as applicable;
Ancillary devices used (e.g., balloon guides, microcatheters) per pass;
Details of the technique used per pass;
Relevant time points during the procedure (e.g. arterial puncture time);
Clot collection for every pass during the procedure; and
Additional interventions performed (if applicable).

In addition, the following were collected throughout the procedure and recorded on the eCRF, including AEs, protocol deviations, and device malfunctions/deficiency. The procedural angiogram was provided to the Independent Core Lab.

### Patient Selection

Subjects presenting with AIS were evaluated and treated by the physician according to the institutional practice prior to enrolment in the study. A subject was enrolled in this study from the point of arrival at medical treatment facility up to the day of discharge or 7 days post-procedure, whichever came first. Screening Logs were utilized at enrolling medical treatment facilities to monitor and ensure consecutive enrolment of patients who meet eligibility criteria.

For Phase I subjects, use of device 200 on the third pass, following angiographic confirmation of an occlusion (mTICI<2b) after two passes of other commercial available stent-retrievers, confirmed subject's eligibility for the study per inclusion/exclusion criteria.

For Phase II subjects, angiographic confirmation of an occlusion (mTICI<2b) after two passes of a commercially available stent-retriever, exclusive of device 200, confirms subject's eligibility for the study per inclusion/exclusion criteria. All assessments and imaging performed for this study are part of SOC for stroke treatment. Informed consent was mandatory and obtained before any data was captured in the eCRF. During the eligibility screening, the investigator performed an initial evaluation of potential study subjects for study eligibility according to inclusion/exclusion criteria. For subjects who met the eligibility criteria and agreed to participate, informed consent was obtained. The investigator, or designee, explained the research study to the subject and/or the legally

Rescue therapy for Phase I subjects included use of another mechanical thrombectomy device during the 4th, or 5th procedural pass (i.e. 1st, 2nd, 3rd pass of device 200), use of aspiration with a pump during the 3rd, 4th, or 5th procedural pass, and use of IA-tPA during the 3rd, 4th, or 5th procedural pass. Rescue therapy for Phase II subjects included use of aspiration with a pump during the 3rd, 4th, or 5th procedural pass and use of IA-tPA during the 3rd, 4th, or 5th procedural pass.

With respect to analysis sets, Modified Intent-to-Treat (mITT) Analysis Sets include for Phase I all subjects who enrolled into the study, and who attempted at least one pass with device 200 (which includes deployment and retrieval with device 200). For Phase II, the mITT Analysis Set consisted of all subjects enrolled into the study, and who attempted at least one pass of any commercially available stents except device 200 (at least one pass after study enrolment includes deployment and retrieval, excluding the initial two unsuccessful passes before study enrolment). Subjects who used device 200 were excluded from the Phase II mITT Analysis Set.

For the mRS analysis set, to define a population more comparable to prior published literature reporting mRS outcomes at 90 days, the mRS Analysis Set included all mITT subjects who meet all of the following criteria: (1) MRI criterion: volume of diffusion restriction visually assessed ≤50 mL, or CT criterion: ASPECTS 6 to 10 on baseline CT or CTA-source images, or, volume of significantly lowered CBV≤50 mL; (2) NIHSS≥8 and <30; and (3) Subjects treated within 6 hours of onset of stroke symptoms (start of treatment defined as groin puncture)

Neurologic evaluation was performed through repeat NIHSS determinations in line with standard of care at 24 hours (−8/+12 hours), 7 days, and 90-day post-procedure. Hospital length of stay, computed tomography (CT) and/or magnetic resonance Imaging (MRI) evidence at each time post-procedure was also carried out to evaluate relevant Adverse Event (AE), which in the study was understood as any untoward medical occurrence, unintended disease or injury, or untoward clinical signs (including abnormal laboratory findings) in subjects, users or other persons, whether or not related to device 200. Any medical condition present at the time the participant was screened or prior to the start of the study procedure was considered as baseline and not reported as an AE. A Serious Adverse Event (SAE) was understood as an AE that a) led to a death, b) led to a serious deterioration in the health of the subject that either resulted in a life-threatening illness or injury, or a permanent impairment of a body structure or a body function, or in-patient hospitalization or prolonged hospitalization, or medical or surgical intervention to prevent life threatening illness or injury or permanent impairment to a body structure or a body function; and led to fetal distress, fetal death or a congenital abnormality or birth defect. An Adverse Device Effect (ADE) was understood as an AE related to device 200.

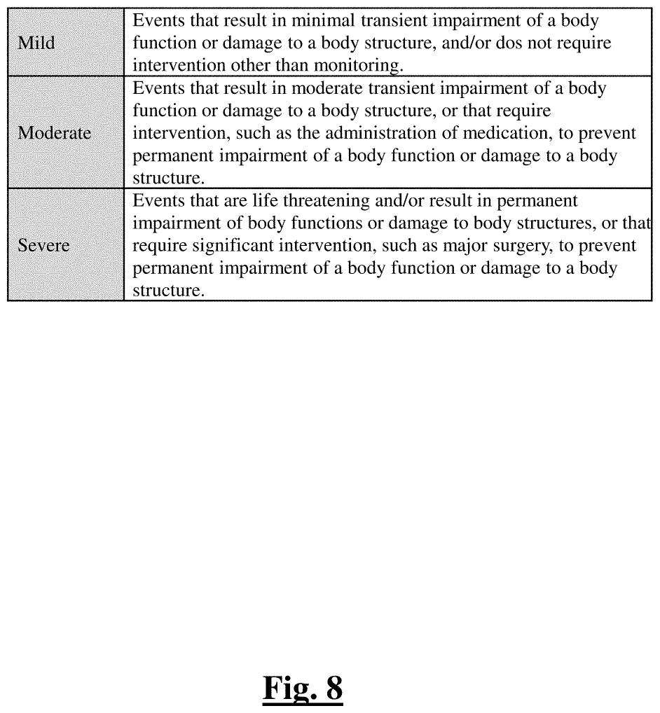

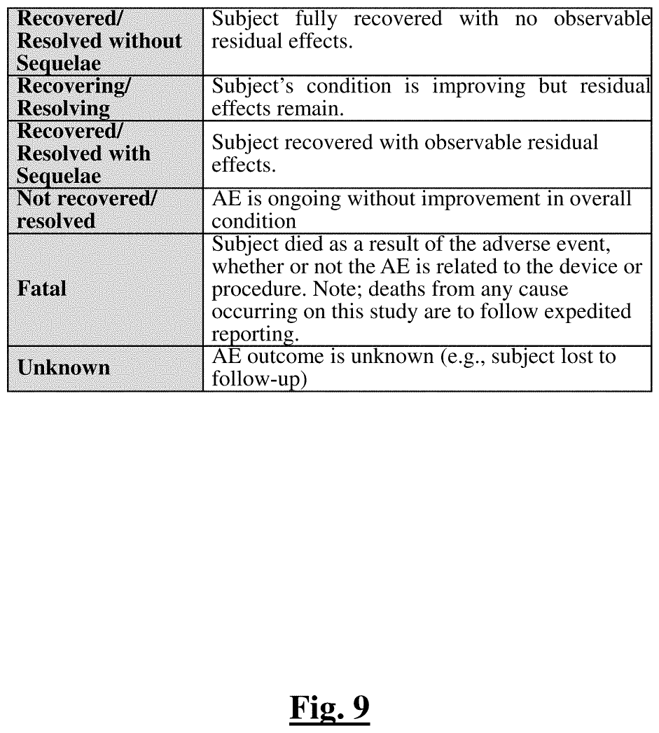

In the study, the investigator also recorded the nature, severity, treatment and outcome of the AE, and determined the relationship to device and procedure and in the case of SADEs whether the event was anticipated or not. The intensity or severity of each AE was assessed according to the following classifications summarized in FIG. 8. The outcome of each AE was also assessed according to classifications summarized in FIG. 9.

Inclusion criteria for the study included the following:
Aged ≥18;
Patient had two passes of another stent-retriever device without achieving mTICI 2b or better and continue to have angiographic confirmation of a Large Vessel Occlusion (LVO) in the same vessel or likely resulting from the same clot;
mRS 0-1 prior to this stroke; and
Prior to entry in this study, Phase I (up to the first 50) patients used device 200 on the third overall pass to attempt revascularization.

Exclusion criteria for the study included the following:
Currently participating in an investigational (drug, device, etc.) clinical trial that may confound study endpoints. Patients in observational, natural history, and/or epidemiological studies not involving intervention were eligible;
Confirmation of positive pregnancy test according to site specific standard of care (e.g. test, verbal communication);
Patients who had direct aspiration via syringe/mechanical pump during the first and/or second pass attempt prior to device 200;
All patients with severe hypertension on presentation (SBP>220 mmHg and/or DBP>120 mm Hg). All patients, in whom intravenous therapy with blood pressure medications was indicated, with hypertension that remained severe and sustained despite intravenous antihypertensive therapy (SBP>185 mmHg and/or DBP>110 mmHg);
Known cerebral vasculitis;
Known cancer with life expectancy less than 12 months;
Stenosis, or any occlusion, in a proximal vessel that requires treatment or prevents access to the site of occlusion;
Intracranial stenosis that prevented access to the site of occlusion;
Computed tomography (CT) or Magnetic Resonance Imaging (MRI) evidence of recent/fresh hemorrhage on presentation;
Baseline CT or MRI showing mass effect or intracranial tumor (except small meningioma);
Evidence of dissection in the extra or intracranial cerebral arteries; and
Occlusions in multiple vascular territories (e.g., bilateral anterior circulation, or anterior/posterior circulation).

### Results of the Study

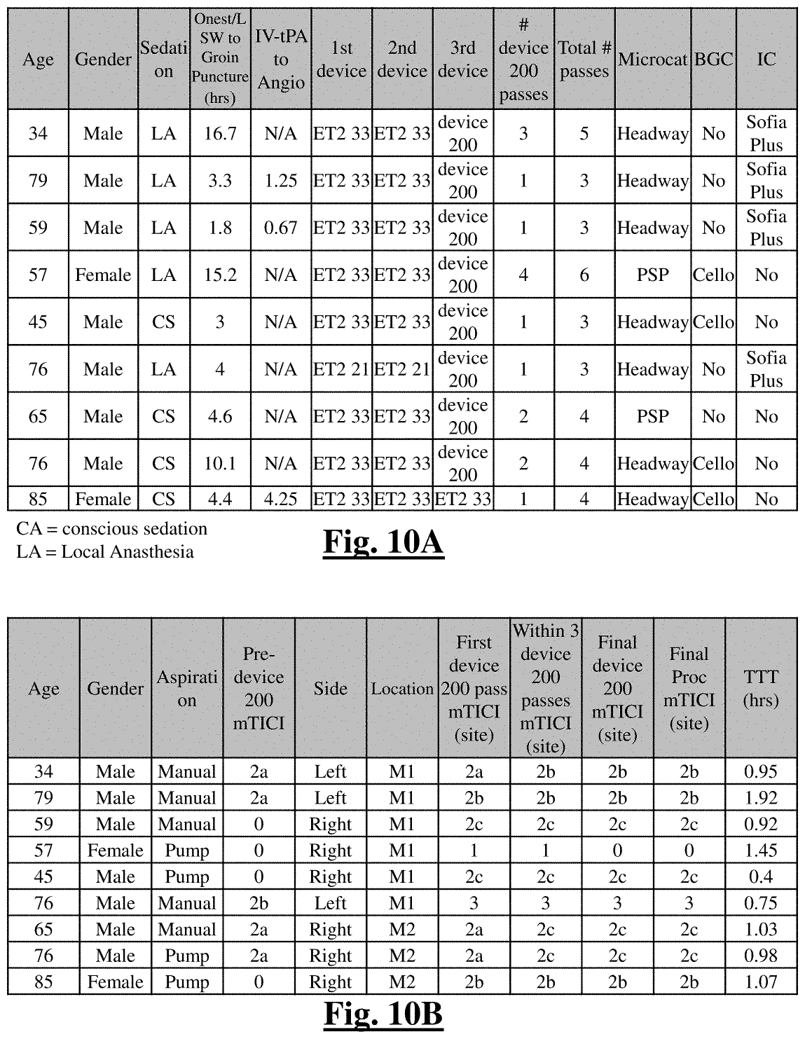

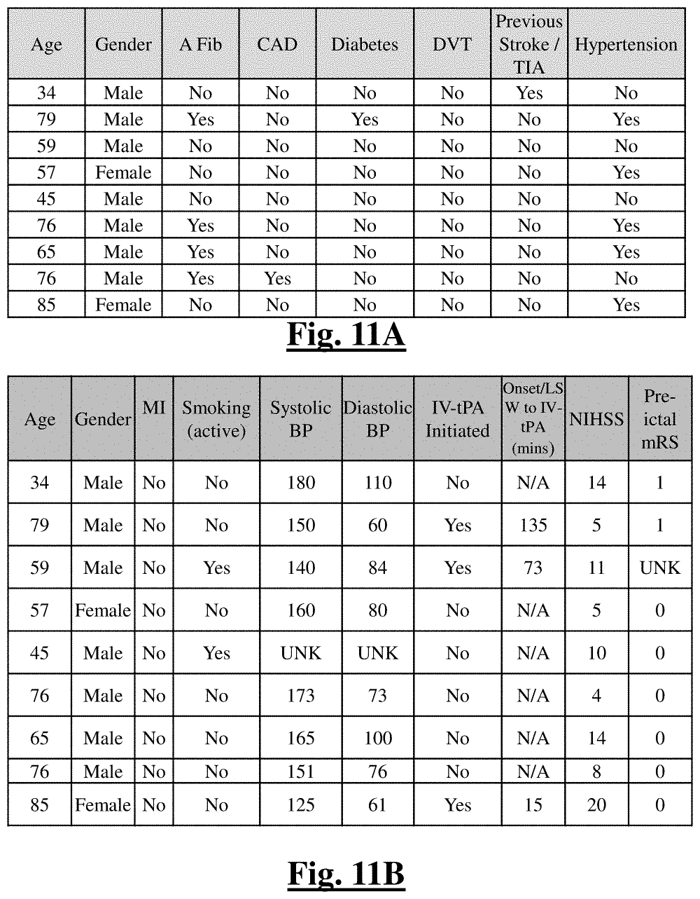

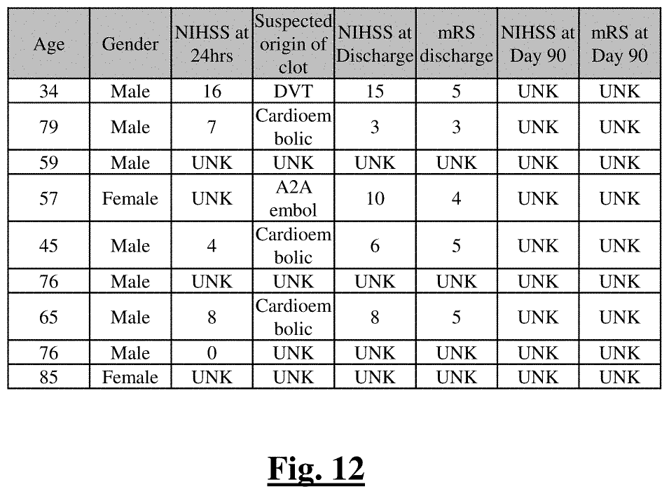

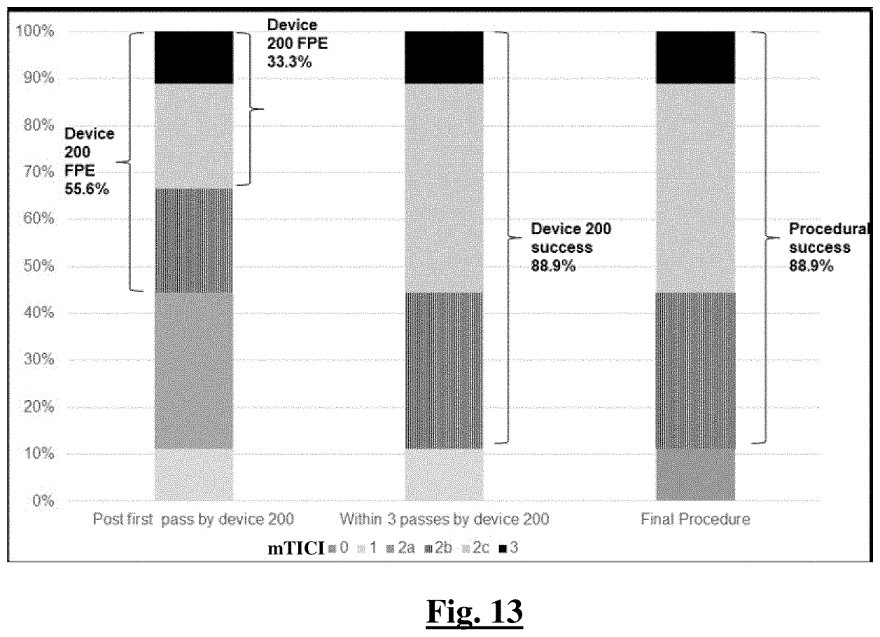

Characteristics of the study are summarized in FIGS. 10A-13. In particular, FIGS. 10A-10B illustrate procedural characteristics. FIGS. 11A-12 illustrate baseline characteristics. FIG. 13 illustrates revascularization results demonstrating that device 200 exhibited approximately 55.6% FPE (mTICI greater than or equal to 2b) post first pass by device 200. Device 200 also exhibited approximately 33.3% FPE (mTICI greater than or equal to 2c) for first pass by device 200. For device 200 being used within 3 passes, device 200 exhibited approximately 88.9% (mTICI greater than or equal to 2b). For final procedural success, device 200 exhibited approximately 88.9% (mTICI greater than or equal to 2b).

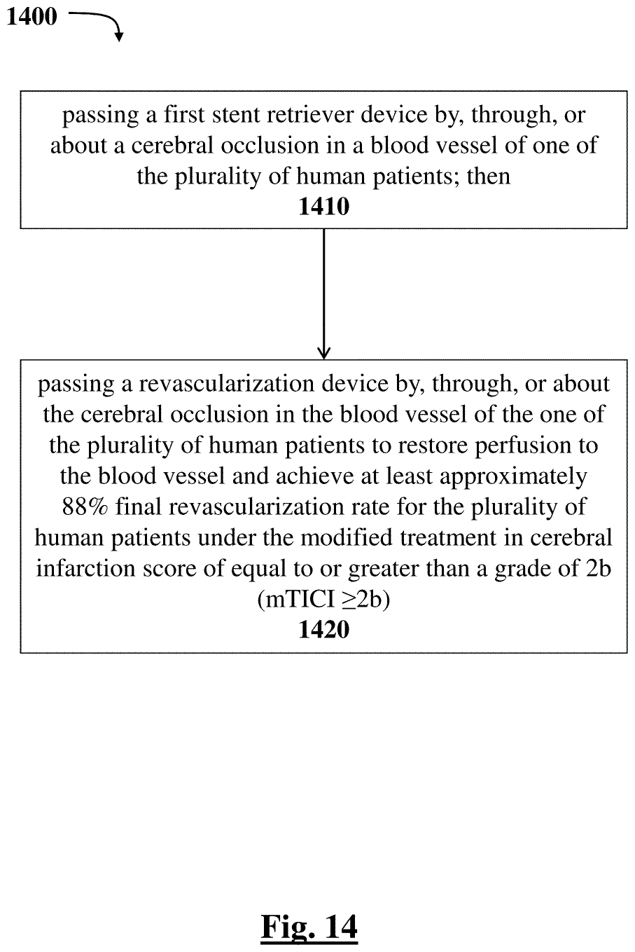

FIG. 14 depicts a method or use 1400 for treating ischemic stroke. The method 1400 can include 1410 passing a first stent retriever device by, through, or about a cerebral occlusion in a blood vessel of one of the plurality of human patients; then 1420 passing a revascularization device by, through, or about the cerebral occlusion in the blood vessel of the one of the plurality of human patients to restore perfusion to the blood vessel and achieve at least approximately 88% final revascularization rate for the plurality of human patients under the modified treatment in cerebral infarction score of equal to or greater than a grade of 2b (mTICI>2b). Method 1400 can end after step 1420. In other embodiments, additional steps according to the examples described above can be performed.

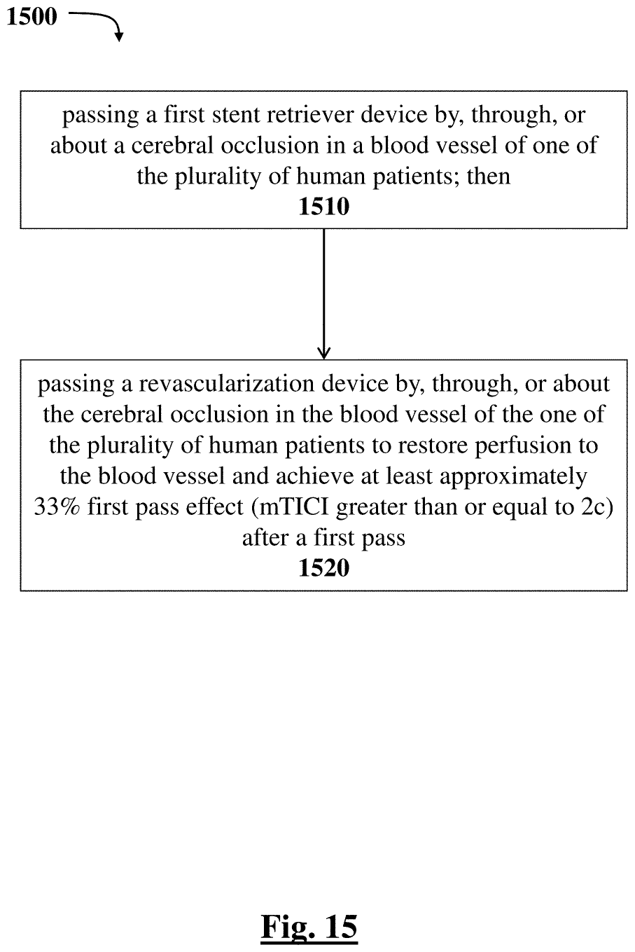

FIG. 15 depicts a method or use 1500 for treating ischemic stroke. The method 1500 can include 1510 passing a first stent retriever device by, through, or about a cerebral occlusion in a blood vessel of one of the plurality of human patients; then 1520 passing a revascularization device by, through, or about the cerebral occlusion in the blood vessel of the one of the plurality of human patients to restore perfusion to the blood vessel and achieve at least approximately 33% FPE (mTICI greater than or equal to 2c) after a first pass. Method 1500 can end after step 1520. In other embodiments, additional steps according to the examples described above can be performed.

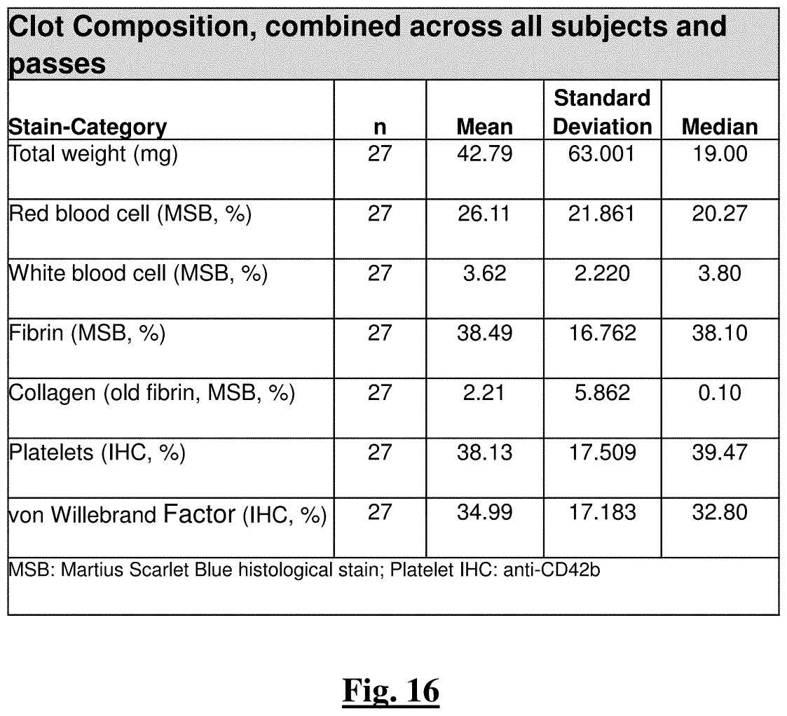

FIG. 16 provides a table summarizing clot composition assessed in patients with challenging clots when device 200 was used after 1 or 2 failed attempts with a comparative device using standard mechanical thrombectomy techniques. Clot compositions in FIG. 16 were analyzed by a blinded central lab. Although not shown in FIG. 16, 54 subjects were enrolled at 11 centers between October 2019 and February 2022. The mean age for the 54 subjects was 71.9±14.09 years. The mean time since onset was 8.4 hours±5.32 hours. In approximately 24.1% of the cases studied, the subjects were administered IV-tPA before mechanical thrombectomy. Device 200 was used in 13/54 subjects after an average of 1.8 passes with comparative device due to failure of the comparative device to remove the clot. When device 200 was used, an average number of 2.2±1.13 passes were performed (4.6±1.72 passes for all devices). Of the 54 subjects enrolled, 27 cases from 8 different centers were analyzed for clot composition per pass. As shown in FIG. 16, the mean total weight of the clot was 42.79 mg±63.001 mg. sing Martius Scarlett Blue (MSB) histological stain, the mean percentage of red blood cells (RBCs) at 26.11%±21.861%, while the mean percentage of fibrin was 38.49%±16.762%. The remaining composition percentage was attributed to white blood cells (mean 3.62%±2.220%) and collagen, or old fibrin (2.21%±5.862%). Platelet IHC and anti-CD42b presented a mean percentage of platelets of 38.13%±17.509% and vol Willebrand Factor of 34.99%±17.13%.

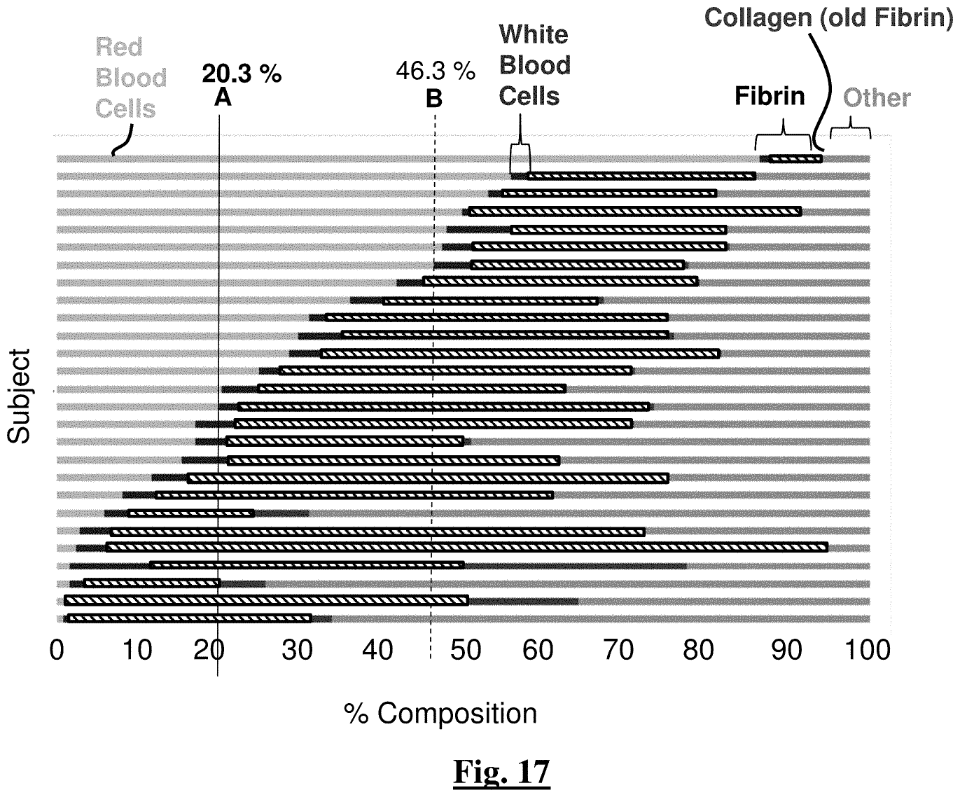

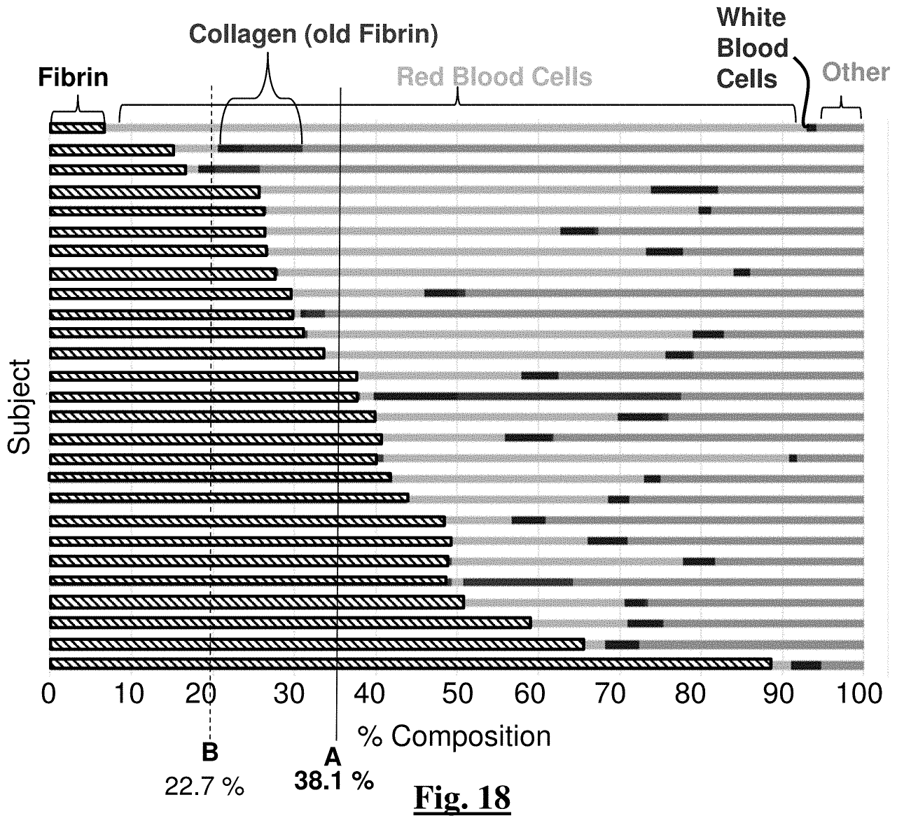

FIGS. 17 and 18 illustrate clot composition across all passes, total composition displayed for each subject. FIG. 17 is clot composition sorted by red blood cell content while FIG. 18 is clot composition sorted by fibrin content. As provided in FIG. 17, organizing each subject by RBC content for the present study generates a median RBC of 20.3%, shown at line marked “A”. Compared to other studies (such as EXCELLENT) comparing device 200 to comparative devices for mechanical thrombectomy without challenging clots, where the median RBC content was identified to be approximately 46.3% and shown at the dashed line marked “B”. Similarly, organizing each subject by fibrin content for the present study generates a median fibrin content of 38.1%, as shown in line “A” of FIG. 18. Compared to other studies (such as EXCELLENT), the median fibrin content was identified to be approximately 22.7%, as marked by the dashed line “B” of FIG. 18.

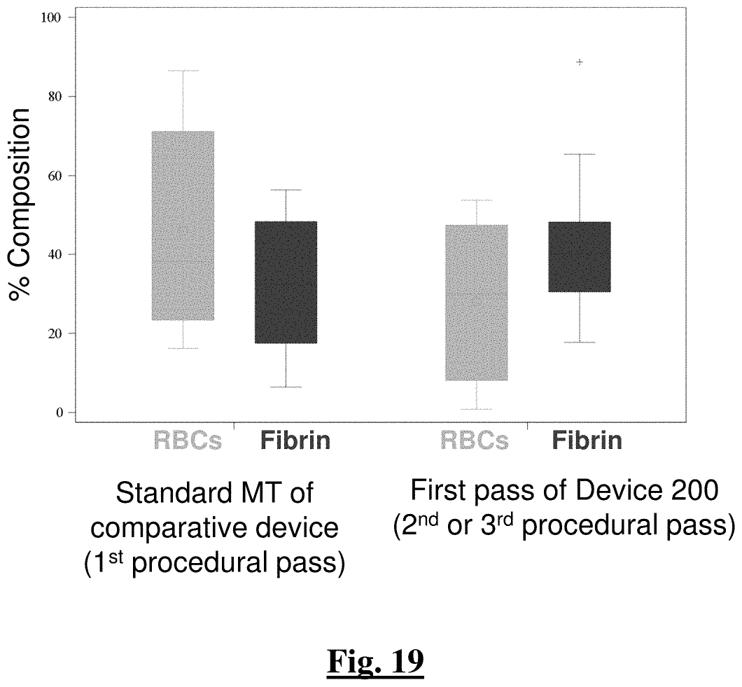

FIG. 19 illustrates composition of clot retrieved in first procedural pass of device 200 compared to clot retrieved with comparative device. The majority of subjects (66.67%; 36/54) had one or more clots retrieved in at least one pass of device 200. The remaining 27 out of 54 subjects had one or more clots retrieved in at least one pass of a comparative device. In pass 2, subjects who switched to device 200 had one or more clots retrieved in approximately 53.8% (7/13) cases compared to 21.9% (9/41) for those that continued with comparative devices beyond two failed passes.

For challenging mechanical thrombectomy cases where device 200 as used after failure of 1 or 2 passes of standard comparative devices, composition analysis of available clots confirmed tough clots having RBC-poor and fibrin-rich compositions. Device 200 was found to be more productive than other devices in removing any clot material in a higher percentage of subjects. First pass of device 200 retrieved clots richer in fibrin and lower in RBC content than fragments retrieved with the first pass of a standard comparative device.

The device 200 and related methods of use of this disclosure demonstrated high rates of substantial reperfusion and functional independence in patients with acute ischemic stroke secondary to large-vessel occlusions. The specific configurations, choice of materials and the size and shape of various elements can be varied according to particular design specifications or constraints requiring a system or method constructed according to the principles of the disclosed technology. Such changes are intended to be embraced within the scope of the disclosed technology. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein.

The following clauses list non-limiting embodiments of the disclosure:

1. A method or use of restoring blood flow in neurovasculature by removing thrombus in a plurality of human patients experiencing ischemic stroke, the method or use including:

passing a first stent retriever device by, through, or about a cerebral occlusion in a blood vessel of one of the plurality of human patients; then

passing a revascularization device by, through, or about the cerebral occlusion in the blood vessel of the one of the plurality of human patients to restore perfusion to the blood vessel and achieve at least approximately 88% final revascularization rate for the plurality of human patients under the modified treatment in cerebral infarction score of equal to or greater than a grade of 2b (mTICI≥2b).

2. The method or use of Clause 1, the step of passing the revascularization device comprises retracting the revascularization device, after being passed by, through or about the cerebral occlusion, while pinching the cerebral occlusion.

3. The method or use of Clause 1, the revascularization device being configured to remove the cerebral occlusion or portions thereof that are fibrin-rich.

4. The method or use of Clause 1, wherein the plurality of human patients including at least approximately 50 patients.

5. The method or use of Clause 1, wherein the plurality of human patients including at least approximately 80 patients.

6. The method or use of Clause 1, further including:
confirming angiographically, after the step of passing the first stent retriever device, that the revascularization rate is less than a grade of 2b (mTICI<2b).

7. The method or use of Clause 6, inclusion criteria for the plurality of human patients including:
Aged ≥18; and
mRS 0-1 prior to a stroke corresponding to the cerebral occlusion.

8. The method or use of Clause 1, exclusion criteria for the plurality of human patients including:
Patients in observational, natural history, and/or epidemiological studies involving intervention;
Confirmation of positive pregnancy test according to site specific standard of care;
Patients who had direct aspiration via syringe/mechanical pump during the first and/or second pass attempt prior to the revascularization device;
All patients with severe hypertension on presentation (SBP>220 mmHg and/or DBP>120 mm Hg). All patients, in whom intravenous therapy with blood pressure medications was indicated, with hypertension that remained severe and sustained despite intravenous antihypertensive therapy (SBP>185 mmHg and/or DBP>110 mmHg);
Known cerebral vasculitis;
Known cancer with life expectancy less than 12 months;
Stenosis, or any occlusion, in a proximal vessel that requires treatment or prevents access to the site of occlusion;
Intracranial stenosis that prevented access to the site of occlusion;
Computed tomography (CT) or Magnetic Resonance Imaging (MRI) evidence of recent/fresh hemorrhage on presentation;
Baseline CT or MRI showing mass effect or intracranial tumor (except small meningioma);
Evidence of dissection in the extra or intracranial cerebral arteries; and
Occlusions in multiple vascular territories (i.e. bilateral anterior circulation, or anterior/posterior circulation).

9. The method or use of Clause 1, the method or use being performed within approximately 6 hours of stroke symptom onset.

10. The method or use of Clause 1, the method or use being performed within approximately 8 hours of stroke symptom onset.

11. The method or use of Clause 1, the method or use being performed within approximately 24 hours of stroke symptom onset.

12. The method or use of Clause 1, the cerebral occlusion being positioned in an internal carotid artery, a M1 segment and/or a M2 segment of a middle cerebral artery, a vertebral artery, or a basilar artery of the patient, the patient being a human.

13. The method or use of Clause 1, the revascularization device having a collapsed delivery configuration and an expanded deployed configuration, the revascularization device including a proximal pinch section including a spiral shape including a spiral pitch; and a distal section including a barrel shape.

14. The method or use of Clause 13,

wherein, in the expanded configuration, the revascularization device comprises peaks of the proximal pinch section are laterally spaced-apart and when under tension,

the method or use including:

pinching the cerebral occlusion between the peaks.

15. The method or use of Clause 13, the proximal pinch section including a plurality of cells defined by struts and crowns connected to corresponding struts and/or crowns, and wherein at least some of the struts and/or crowns of the clot engaging section are aligned with the wave-like form to enhance embedding of clot.

16. The method or use of Clause 13, the proximal pinch section including one or more clot gripping features.

17. The method or use of Clause 13, the proximal pinch section is substantially curvilinear in the collapsed and expanded configurations.

18. The method or use of Clause 13, the proximal pinch section comprises a transverse cross section between peaks including both flat and curved sections.

19. The method or use of Clause 13, the proximal pinch section comprises a flat shape in transverse cross section.

20. A method or use of restoring blood flow in neurovasculature by removing thrombus in a plurality of human patients experiencing ischemic stroke, the method or use including:

passing a first stent retriever device by, through, or about a cerebral occlusion in a blood vessel of one of the plurality of human patients; then

passing a revascularization device by, through, or about the cerebral occlusion in the blood vessel of the one of the plurality of human patients to restore perfusion to the blood vessel and achieve at least approximately 33% FPE (mTICI greater than or equal to 2c) after a first pass.

21. A revascularization device for treating ischemic stroke, the device including a collapsed delivery configuration and an expanded deployed configuration, the device including:

a proximal pinch section including a spiral shape including a spiral pitch; and

a distal section including a barrel shape

the device, after a first stent retriever device is passed by, through, or about a cerebral occlusion in a blood vessel of a human patient, being configured to achieve at least approximately 88% final revascularization rate under the modified treatment in cerebral infarction score of equal to or greater than a grade of 2b (mTICI≥2b).

22. The device of Clause 21, the revascularization device being configured to remove the cerebral occlusion or portions thereof that are fibrin-rich.

23. The device of Clause 21, the cerebral location of the human patient being located in one of the following locations: a carotid artery, a M1 middle cerebral artery, a M2 middle cerebral artery, a basilar artery, and a vertebral artery.

24. The device of Clause 21, wherein, in the expanded configuration, the revascularization device comprises peaks of the proximal pinch section are laterally spaced-apart and when under tension the proximal pinch section is configured to pinch the cerebral occlusion between the peaks.

25. The device of Clause 21, further including:

a shaft extending between a proximal end and a distal end;

the proximal pinch section coupled to the distal end of the shaft.

26. The device of Clause 21, the proximal pinch section including a plurality of cells defined by struts and crowns connected to corresponding struts and/or crowns, and wherein at least some of the struts and/or crowns of the clot engaging section are aligned with the wave-like form to enhance embedding of clot.

27. The device of Clause 21, the proximal pinch section including one or more clot gripping features.

28. The device of Clause 21, the proximal pinch section is substantially curvilinear in the collapsed and expanded configurations.

29. The device of Clause 21, the proximal pinch section comprises a transverse cross section between peaks including both flat and curved sections.

30. The device of Clause 21, the proximal pinch section comprises a flat shape in transverse cross section.

31. The device of Clause 21, the device configured for use within approximately 6 hours of stroke symptom onset.

32. The device of Clause 21, the device configured for use within approximately 8 hours of stroke symptom onset.

33. The device of Clause 21, the device configured for use within approximately 24 hours of stroke symptom onset.

34. A revascularization device for treating ischemic stroke, the device including a collapsed delivery configuration and an expanded deployed configuration, the device including:

a proximal pinch section including a spiral shape including a spiral pitch; and

a distal section including a barrel shape

the device, after a first stent retriever device is passed by, through, or about a cerebral occlusion in a blood vessel of a human patient, being configured to achieve at least approximately 33% FPE (mTICI greater than or equal to 2c) after a first pass.

35. The device of Clause 34, the revascularization device being configured to remove the cerebral occlusion or portions thereof that are fibrin-rich.

36. The device of Clause 34, the cerebral location of the human patient being located in one of the following locations: a carotid artery, a M1 middle cerebral artery, a M2 middle cerebral artery, a basilar artery, and a vertebral artery.

37. The device of Clause 34, wherein, in the expanded configuration, the revascularization device comprises peaks of the proximal pinch section are laterally spaced-apart and when under tension the proximal pinch section is configured to pinch the cerebral occlusion between the peaks.

38. The device of Clause 34, further including:

a shaft extending between a proximal end and a distal end;

the proximal pinch section coupled to the distal end of the shaft.

39. The device of Clause 34, the proximal pinch section including a plurality of cells defined by struts and crowns connected to corresponding struts and/or crowns, and wherein at least some of the struts and/or crowns of the clot engaging section are aligned with the wave-like form to enhance embedding of clot.

40. The device of Clause 34, the proximal pinch section including one or more clot gripping features.

41. The device of Clause 34, the proximal pinch section is substantially curvilinear in the collapsed and expanded configurations.

42. The device of Clause 34, the proximal pinch section comprises a transverse cross section between peaks including both flat and curved sections.

43. The device of Clause 34, the proximal pinch section comprises a flat shape in transverse cross section.

44. The device of Clause 34, the device configured for use within approximately 6 hours of stroke symptom onset.

45. The device of Clause 34, the device configured for use within approximately 8 hours of stroke symptom onset.

46. The device of Clause 34, the device configured for use within approximately 24 hours of stroke symptom onset.

What is claimed is:

1. A method or use of restoring blood flow in neurovasculature by removing thrombus in a plurality of human patients experiencing ischemic stroke, the method or use comprising:
passing a first stent retriever device by, through, or about a cerebral occlusion in a blood vessel of one of the plurality of human patients; then
passing a revascularization device by, through, or about the cerebral occlusion in the blood vessel of the one of the plurality of human patients to restore perfusion to the blood vessel and achieve at least approximately 88% final revascularization rate for the plurality of human patients under the modified treatment in cerebral infarction score of equal to or greater than a grade of 2b (mTICI≥2b).

2. The method or use of claim 1, the step of passing the revascularization device comprises retracting the revascularization device, after being passed by, through or about the cerebral occlusion, while pinching the cerebral occlusion.

3. The method or use of claim 1, the revascularization device being configured to remove the cerebral occlusion or portions thereof that are fibrin-rich.

4. The method or use of claim 1, wherein the plurality of human patients comprising at least approximately 50 patients.

5. The method or use of claim 1, wherein the plurality of human patients comprising at least approximately 80 patients.

6. The method or use of claim 1, further comprising:
confirming angiographically, after the step of passing the first stent retriever device, that the revascularization rate is less than a grade of 2b (mTICI<2b).

7. The method or use of claim 6, inclusion criteria for the plurality of human patients comprising:
Aged ≥18; and
mRS 0-1 prior to a stroke corresponding to the cerebral occlusion.

8. The method or use of claim 1, exclusion criteria for the plurality of human patients comprising:
Patients in observational, natural history, and/or epidemiological studies involving intervention;
Confirmation of positive pregnancy test according to site specific standard of care;
Patients who had direct aspiration via syringe/mechanical pump during the first and/or second pass attempt prior to the revascularization device;
All patients with severe hypertension on presentation (SBP>220 mmHg and/or DBP>120 mm Hg). All patients, in whom intravenous therapy with blood pressure medications was indicated, with hypertension that remained severe and sustained despite intravenous antihypertensive therapy (SBP>185 mmHg and/or DBP>110 mmHg);
Known cerebral vasculitis;
Known cancer with life expectancy less than 12 months;
Stenosis, or any occlusion, in a proximal vessel that requires treatment or prevents access to the site of occlusion;
Intracranial stenosis that prevented access to the site of occlusion;
Computed tomography (CT) or Magnetic Resonance Imaging (MRI) evidence of recent/fresh hemorrhage on presentation;
Baseline CT or MRI showing mass effect or intracranial tumor (except small meningioma);
Evidence of dissection in the extra or intracranial cerebral arteries; and
Occlusions in multiple vascular territories (i.e. bilateral anterior circulation, or anterior/posterior circulation).

9. The method or use of claim 1, the method or use being performed within approximately 6 hours of stroke symptom onset.

10. The method or use of claim 1, the method or use being performed within approximately 8 hours of stroke symptom onset.

11. The method or use of claim 1, the method or use being performed within approximately 24 hours of stroke symptom onset.

12. The method or use of claim 1, the cerebral occlusion being positioned in an internal carotid artery, a M1 segment and/or a M2 segment of a middle cerebral artery, a vertebral artery, or a basilar artery of the patient, the patient being a human.

13. The method or use of claim 1, the revascularization device having a collapsed delivery configuration and an expanded deployed configuration, the revascularization device comprising a proximal pinch section comprising a spiral shape comprising a spiral pitch; and a distal section comprising a barrel shape.

14. The method or use of claim 13,
wherein, in the expanded configuration, the revascularization device comprises peaks of the proximal pinch section are laterally spaced-apart and when under tension,
the method or use comprising:
pinching the cerebral occlusion between the peaks.

15. The method or use of claim 13, the proximal pinch section comprising a plurality of cells defined by struts and crowns connected to corresponding struts and/or crowns, and wherein at least some of the struts and/or crowns of the clot engaging section are aligned with the wave-like form to enhance embedding of clot.

16. The method or use of claim 13, the proximal pinch section comprising one or more clot gripping features.

17. The method or use of claim 13, the proximal pinch section is substantially curvilinear in the collapsed and expanded configurations.

18. The method or use of claim 13, the proximal pinch section comprises a transverse cross section between peaks comprising both flat and curved sections.

19. The method or use of claim 13, the proximal pinch section comprises a flat shape in transverse cross section.

20. A method or use of restoring blood flow in neurovasculature by removing thrombus in a plurality of human patients experiencing ischemic stroke, the method or use comprising:
passing a first stent retriever device by, through, or about a cerebral occlusion in a blood vessel of one of the plurality of human patients; then
passing a revascularization device by, through, or about the cerebral occlusion in the blood vessel of the one of the plurality of human patients to restore perfusion to the blood vessel and achieve at least approximately 33% FPE (mTICI greater than or equal to 2c) after a first pass.
